(19) United States
(12) Patent Application Publication
(10) Pub. No.: US 2005/0260285 A1
(43) Pub. Date: Nov. 24, 2005

(54) Healthy prostate formula

(76) Inventor: Giovina DiMateeo-Leggio, Aventura, FL (US)

Correspondence Address: GIOVINA INTERNATIONAL INC. OF AMERICA, SUITE 807, 19495 BISCAYNE BLVD., AVENTURA, FL, 33180, US

(21) Appl. No.: 10/628,345

(22) Filed: May 21, 2004

(51) Int. Cl. A61K 35/78

(52) U.S. Cl. 424/725

(57) ABSTRACT

The supplements are a combination of natural herbs, vitamins and minerals used to help alleviate symptoms and risks related to benign prostatic hyperplasia (enlarged prostate). They are packaged in bottles of 150 tablets. The supplements are in the form of tablets of which five tablets daily are taken orally.

NATURAL HEALTH PROSTATE
SUPPLEMENT FACTS
Serving Size 5 tablets

| Amount per serving | | | % Daily Value |
|---|---|---|---|
| Vitamin C (ascorbic acid) | 500 | mg | 833% |
| Vitamin D (Cholecalciferol) | 200 | IU | 50% |
| Vitamin E (DL-Alpha Tocopheryl | 400 | IU | 1333% |
| Succinate) | | | |
| Vitamin B-6 (Pyridoxine hcl) | 100 | mg | 5000% |
| Ca (Dicalcium Phosphate) | 100 | mg | 10% |
| Magnesium (Oxide) | 200 | mg | 50% |
| Chromium (Chromium A A Chelate) | 100 | mcg | 83% |
| Zinc (Zinc Oxide) | 20 | mg | 133% |
| Selenium (Selenium A A Chelate) | 200 | mcg | 286% |
| Indole-3Carbinol | 300 | mg | |
| Forslean 10% | 100 | mg | |
| Pregnenelone | 50 | mg | |
| Lycopene (5%) | 10 | mg | |
| Saw Palmetto 4:1 EXT | 250 | mg | |
| Pygeum 4:1 EXT | 100 | mg | |
| Nettle Leaf powder (Uritica Dioica) | 500 | mg | |
| Rosemary Leaf 4:1 EXT | 450 | mg | |
| Sterols (Beta-Sitosterol) | 10 | mg | |
| Green Tea EXT | 300 | mg | |
| Curcumin powder | 500 | mg | |
| Bioperine ( Piper Nigrum) | 5 | mg | |
| Fatty Acids ( Omega 3 powder) | 50 | mg | |
| GLA (Borage Oil 10% powder) | 50 | mg | |
| CLA (CLA 50% powder) | 250 | mg | |
| L-Glycine | 100 | mg | |
| L-Glutamine | 450 | mg | |

% Daily Value not to established
Directions:
Take 5 tablets 1 daily. Preferably with meals.
150 TABS PER BOTTLE
Other ingredients:
Fribosol, Stearic Acid, Magnesium Stearate, Silicon Dioxide, Hydroxypropyl, Cellulose, Hydroxypropyl Methylcellulose

### DETAILED DESCRIPTION OF HEALTHY PROSTATE FORMULA

Healthy Prostate Formula

The supplements are a combination of natural herbs, vitamins and minerals that help alleviate symptoms and risks related to benign prostatic hyperplasia (enlarged prostate). Healthy Prostate helps alleviate symptoms such as: urinary hesitancy, nocturia, incontinence, urinary retention, pain or burning sensation during urination or ejaculation, and pain or stiffness in the lower back, hips or upper thighs.

Healthy Prostate Formula is packaged in blue PETE bottles of 150 tablets with a black child resistant cap and silver foil label with blue print. The supplements are in the form of tablets of which five tablets daily are to be taken orally.

1-6. (canceled)

7. A method of inducing weight loss and/or fat loss in humans in need thereof, wherein a composition comprising the administration of effective amounts of natural herbs and dietary supplements such as: Vitamin C (500 mg), Vitamin B6 (15 mg), chromium (100 mcg), forslean (100 mg), garcinia gambogla (1000 mg), green tea extract (500 mg), eletheurococcus senticosus (500 mg), guggul extract (500 mg), bitter orange (50 mg), kelp (250 mg), bladderwrack (300 mg), dandelion (500 mg), kola nut (200 mg), guarana (250 mg), L'carnitine (500 mg), dL-phenylalanine (500 mg), L'tyrosine (500 mg), and conjugated Linolenic Acid (1000 mg); said amounts being sufficient to stimulate metabolic function, boost energy, control carbohydrate cravings and consume stored body fat.

8. A method wherein, according to claim 7 said ingredients are compressed into tablet form and administered orally.

9. A method of claim 8 wherein a total of 6 tablets are taken orally daily one half hour before meals with a full glass of water.

10. A method of claim 7 wherein said ingredients are for treating over-weight or obesity, symptoms comprised of excess volume of body fat as compared to normal and healthy ranges publicly stipulated.

11. A method of claim 10 wherein said ingredients provoke a normal body weight.

12. A method of Hormone Replacement Therapy reducing symptoms associated with menopause, such as hot flashes, edema, fatigue, mental confusion and forgetfulness, hormone unbalance, bone degeneration and depression, which comprises administering to a human perimenopausal or menopausal female, comprising of the administration of this composition of effective (+) amounts of natural herbs and dietary supplements in the amounts of: Vitamin C (250 mg), vitamin D (250 IU), vitamin E (150 IU), Vitamin B6 (50 mg), Pantothenic Acid (125 mg), calcium (250 mg), magnesium (75 mg), chromium (50 mcg), CoQ10 (50 mg), Biaflavinoids (250 mg), dandelion root (250 mg), coleus forskoli root (50 mg), Don Quai (125 mg), green tea extract (150 mg), grape seed extract (150 mg), uva ursi (250 mg), howthorn extract (50 mg), skullcap (150 mg) larex immune enhancer AG (250 mg), chasteberry (250 mg), black cohosh (250 mg), ginkgo biloba extract (30 mg), Mexican wild yam (30 mg), curcumin (250 mg), bioperine (5 mg), CoQ10 (50 mg), soy isoflavone extract (100 mg), biaflovinoids (250 mg), gamma-oryzanol (75 mg), CLA (250 mg), and L-glutamine (250 mg); said amounts being sufficient to provoke a substantial decrease of menopause symptoms and cause normal: body temperatures, energy levels, water volume, mental clarity and memory, youthful hormone balance, healthy bones and emotional disposition.

13. A method of claim 12 wherein menopausal symptoms: atrophic vaginitis, bladder infections, breast tenderness and PMS, cold hands and feet, depression, endometriosis, fatigue, fibrocystic breasts, forgetfulness and lack of concentration, headaches, heart disease, hot flashes, insomnia, joint and muscle pains, low libido, mood swings, anxiety and irritability, night sweats, osteoporosis, uterine cramps, and weight gain; are significantly reduced or eliminated without the risk of degenerative disease (stroke, heart disease, cancer) provoked by conventional hormone replacement treatments.

14. A method of claim 12 wherein (+) natural herbal and dietary supplement Ingredients are administered orally as a tablet or a capsule.

15. Method of claim 14 wherein a total of 6 tablets are taken daily administered orally.

16. A method of maintaining healthy prostate function, which comprises administering to a human middle aged male, a composition of effective (+) amounts of natural herbs and dietary supplements in the amounts of: Vitamin C (500 mg), vitamin D (200 IU), vitamin E (400 IU), vitamin B-6 (100 mg), Calcium (100 mg), Magnesium (200 mg), Chromium (100 mcg) zinc (20 mg), selenium (200 mcg), Indole-3Carbinol (300 mg), forslean (100 mg) pregnenelone (50 mg), lycopene (10 mg), saw palmetto (250 mg), pygeum (100 mg), nettle leaf powder (500 mg), sterols (10 mg), green tea extract (300 mg), curcumin powder (500 mg), bioperine (5 mg), fatty acids (50 mg), GLA (50 mg), CLA (250 mg), L-Gluthamine (450 mg), L'glycine (100 mg), said amounts being sufficient to provoke healthy prostate function and for the treatment of benign prostatic hyperplasia; without altering PSA concentration, thus poses no risk of undetected prostate cancer.

17. A method of claim 16 wherein symptoms of benign prostatic hyperplasia; urinary hesitancy, weak or interrupted urine flow, nocturia, pain or burning sensation during urination or ejaculation, urinary retention, increased urinary frequency, incontinence, pain or stiffness in the lower back, hips, or upper thighs; are significantly reduced and/or eliminated.

18. A method of claim 17 wherein regular prostate function is restored to normal urine flow and frequency, blocks estrogen binding, decreases prolactin production, enhances the immune function, decreases bladder hyper contractility and decompensation.

19. A method of claim 18 wherein relief of symptoms of benign prostatic hyperplasia, results closely simulate actions produced by 5 alpha reductase inhibitors and alpha-adrenergic blockers.

20. The method of claim 16 wherein (+) Ingredients are administered orally as a tablet.

21. Method of claim 20 wherein a total of 5 tablets are taken daily administered orally.

22. A method of reducing symptoms associated with cellulite deposits, which comprises administering to a human usually a female, a composition of effective (+) amounts of natural herbs and dietary supplements in the amounts of Vitamin C (250 mg), Vitamin B6 (15 mg), Vitamin E (150 IU), Chromium (10 mcg), forslean (100 mg), ginkgo biloba (20 mg), bladderwrack (50 mg), gotu kola extract (100 mg), cinnamon (50 mg), grape seed extract (150 mg), kelp (100 mg), evening primerose (200 mcg), sage leaf (100 mg), uva ursi (200 mg), lecithin (300 mg), senna leaf (50 mg), cascara sagrada (50 mg), 5-hydroxy tryptopan (1000 mg), dandelion (100 mg), bioperine (5 mg), L'carnitine (100 mg), and conjugated linolenic acid (1000 mg); said amounts being sufficient to provoke a substantial decrease cellulite fat deposits on the human body.

23. A method of claim 22 wherein cellulite symptoms: tissue damaged by the malfunction of the organic systems, inefficient waste elimination by the lymphatic system and the blood stream, deficient blood microcirculaiton, nodule growth stimulation (fatty cells that have grown in size due to the accumulation of toxins, water and fat), lack of oxygen circulation at the cell level, and formation of fibroblasts (fiber around the fatty tissue); are significantly reduced or eliminated with effective amounts of said ingredients.

24. A method of claim 23 wherein lipotropic, detoxifying and lymphatic activity is substantially increased to promote proper circulation and elimination of toxic waste.

25. A method of claim 22 wherein thermogenic and vasodilatation components in effective quantities stimulate the dissipation of cellulite.

26. A method of claim 22 wherein effective quantities of said ingredients stimulate circulation, metabolism and purging activity.

27. A method of claim 22 wherein elimination of fat and fibroblasts is facilitated.

28. The method of claim 22 wherein the appropriate blend of (+) natural herbal and dietary supplement Ingredients are administered orally as a capsule administered orally.

29. Method of claim 28 wherein a total of 6 tablets are taken daily administered orally.
